(19) United States
(12) Patent Application Publication
(10) Pub. No.: US 2021/0121492 A1
(43) Pub. Date: Apr. 29, 2021

(54) METHODS FOR THE TREATMENT AND PREVENTION OF RENAL DISORDERS AND FATTY LIVER DISORDERS

(71) Applicant: Janssen Pharmaceutica NV, Beerse (BE)

(72) Inventor: Keith S. Usiskin, Mendham, NJ (US)

(21) Appl. No.: 17/141,248

(22) Filed: Jan. 5, 2021

Related U.S. Application Data
(63) Continuation of application No. 16/040,580, filed on Jul. 20, 2018.
Application No. 16/040,580 is a continuation of application No. 15/112,786, filed on Jul. 20, 2016, now abandoned.
(60) Provisional application No. 61/948,882, filed on Mar. 6, 2014.
(60) Provisional application No. 61/934,003, filed on Jan. 31, 2014.

(51) Int. Cl. A61K 31/7042; A61K 45/06; A61K 31/401; A61K 31/4164; A61K 31/4439; A61K 31/41; A61K 31/4178; A61K 31/4184; A61K 31/55; A61K 38/05

(52) U.S. Cl. CPC A61K 31/7042; A61K 45/06; A61K 31/401; A61K 31/4164; A61K 38/05; A61K 31/41; A61K 31/4178; A61K 31/4184; A61K 31/55; A61K 31/4439

(57) ABSTRACT

The present invention is directed to methods for treating, delaying, slowing the progression of and/or preventing disorders comprising administering to a subject in need thereof a therapeutically effective amount of co-therapy comprising, consisting or consisting essentially of (a) canagliflozin and (b) one or more ACE inhibitors or one or more ARBs or one or more PPAR-gamma agonists; and to methods for treating, delaying, slowing the progression of and/or preventing fatty liver disorders (for example, NASH or NAFLD), comprising administering to a subject in need thereof a therapeutically effective amount of canagliflozin.

### CROSS REFERENCE TO RELATED APPLICATIONS

This application claims the benefit of U.S. Provisional Application 61/934,003, filed on Jan. 31, 2014, and U.S. Provisional Application 61/948,882, filed on Mar. 6, 2014, which are incorporated by reference herein in their entireties.

### FIELD OF THE INVENTION

The present invention is directed to methods for treating, delaying, slowing the progression of and/or preventing renal diseases, comprising administering to a subject in need thereof a therapeutically effective amount of co-therapy comprising, consisting or consisting essentially of (a) canagliflozin and (b) one or more ACE inhibitors or one or more ARBs.

The present invention is further directed to methods for treating, delaying, slowing the progression of and/or preventing fatty liver disorders (for example NAFLD or NASH), comprising administering to a subject in need thereof a therapeutically effective amount of canagliflozin. The present invention is further directed to methods for treating, delaying, slowing the progression of and/or preventing fatty liver disorders (for example, NAFLD or NASH), comprising administering to a subject in need thereof a therapeutically effective amount of co-therapy comprising, consisting or consisting essentially of (a) canagliflozin and (b) one or more ACE inhibitors or one or more ARBs or one or more PPAR-gamma agonists.

### BACKGROUND OF THE INVENTION

Kidneys are bean-shaped organs, located near the middle of the back. Inside each kidney about a million tiny structures called nephrons filter blood. They remove waste products and extra water, which become urine. Damage to the nephrons represents an important form of kidney disease. This damage may leave kidneys unable to remove wastes. Some damage, e.g. damage related to hyperfiltration can occur slowly over years, initially often without obvious symptoms.

The ‘hyperfiltrative hypothesis’ implies that the excess demand on a limited renal reserve produces adaptive and ultimately pathologic changes in the kidney which finally lead to ‘nephron exhaustion’. At the single-nephron level, hyperfiltration is hypothesized to be an early link in the chain of events that lead from intraglomerular hypertension to albuminuria and, subsequently, to reduced Glomerular Filtration Rate (GFR). Based on this hyperfiltration therefore represents a risk for subsequent renal injury and could be classified as an early manifestation of renal pathology often referred to as the hyperfiltrative stage. Such renal hyperfiltration can lead to early glomerular lesions and to microalbuminuria, which itself can lead to macroalbuminuria and to end-stage renal disease.

The influence of hyperfiltration on renal function decline has been most thoroughly evaluated in kidney transplant recipients and donors, and in patients with a single kidney removed for acquired renal disease, but also in patients with diabetes mellitus (Magee et al. Diabetologia 2009; 52: 691-697). In theory, any reduction in functional nephron number will lead to adaptive glomerular hyperfiltration whether induced genetically, surgically, or by acquired renal disease. Moreover, hyperfiltration has been shown to occur in certain pathophysiologic conditions even when renal mass is intact, e.g. in diabetes. Therefore, there is a medical need for interventions with a good efficacy with regard to renal hyperfiltrative injury.

Creatinine is a breakdown product of creatine phosphate in muscle tissue, and is usually produced at a constant rate in the body. Serum creatinine is an important indicator of renal health, because it is an easily measured byproduct of muscle metabolism that is excreted unchanged by the kidneys. Creatinine is removed from the blood chiefly by the kidneys, primarily by glomerular filtration, but also by proximal tubular secretion. Little or no tubular reabsorption of creatinine occurs. If the filtration in the kidney is deficient, creatinine blood levels rise. Therefore, creatinine levels in blood and urine may be used to calculate the creatinine clearance (CrCl), which correlates with the glomerular filtration rate (GFR). Blood creatinine levels may also be used alone to estimate the GFR (eGFR). The GFR is clinically important because it is a measurement of renal function. An alternate estimation of renal function can be made when interpreting the blood (plasma) concentration of creatinine along with that of urea. The BUN-to-creatinine ratio (the ratio of blood urea to creatinine) can indicate other problems besides those intrinsic to the kidney; for example, a urea level raised out of proportion to the creatinine may indicate a pre-renal problem such as volume depletion.

A rise in blood creatinine level is observed only with marked damage to functioning nephrons. An estimation of kidney function is given by calculating the estimated glomerular filtration rate (eGFR). eGFR can be accurately calculated using serum creatinine concentration. The typical human reference ranges for serum creatinine are 0.5 to 1.0 mg/dl (about 45-90 μmol/l) for women and 0.7 to 1.2 mg/dl (60-110 μmol/l) for men. The trend of serum creatinine levels over time is generally more important than absolute creatinine level.

Creatinine levels may increase modestly when an ACE inhibitor (ACEi) or angiotensin II receptor antagonist (or angiotensin receptor blocker, ARB) is taken. Using both an ACE inhibitor and ARB concomitantly will increase creatinine levels to a greater degree than either of the two drugs would individually. An increase of <30% is to be expected with ACE inhibitor or ARB use.

Albuminuria is a condition, where albumin is present in the urine. In healthy individuals, albumin is filtered by the kidneys. When the kidneys do not properly filter large molecules (such as albumin) from the urine, albumin is excreted in urine and is typically a sign of kidney damage or excessive salt intake. Albuminuria can also occur in patients with long-standing diabetes mellitus, either Type I (1) or Type II (2) diabetes mellitus. Urine albumin may be measured by dipstick or as direct measure of the amount of protein excreted in total volume of urine collected over a 24 hour period

Microalbuminuria, occurs when the kidney leaks small amounts of albumin into the urine, as a result of an abnormally high permeability for albumin in the renal glomerulus. Microalbuminuria as a condition of diabetic nephropathy is indicated when urine albumin levels are in the range of 30 mg to 300 mg in a 24 hour period.

An alternate measure of microalbuminuria is creatinine levels and the ratio of albumin to creatinine in serum. The albumin/creatinine ratio (ACR) and microalbuminuria are defined as ACR mg/mmol (female) or ≥2.5 mg/mmol (male), or, with both substances measured by mass, as an ACR between 30 μg albumin/mg creatinine and 300 μg albumin/mg creatinine.

Microalbuminuria may be an important prognostic marker for the development and progression of kidney disease, particularly in patients with diabetes mellitus or hypertension. Microalbuminuria is also an indicator of subclinical cardiovascular disease, a marker of vascular endothelial dysfunction and a risk factor for venous thrombosis.

Diabetic nephropathy is one of the microvascular complications of diabetes mellitus and is characterized by persistent albuminuria and a progressive decline in renal function. Hyperglycemia is an important contributor to the onset and progression of diabetic nephropathy.

The clinical progression of diabetic nephropathy in patients with T1DM (Type 1 Diabetes Mellitus) is well characterized. Initially, hyperfiltration accompanied by increases in glomerular filtration rate (GFR) and increased renal plasma flow is seen. A meta-analysis found that the presence of hyperfiltration in patients with T1DM more than doubled the risk of developing micro- or macroalbuminuria. This phase is followed by reductions in GFR and the development of microalbuminuria, defined as urinary albumin excretion of ≥30 mg/day (or 20 μg/min) and <300 mg/24 h (or <200 μg/min), which may be accompanied by increases in blood pressure. Later in the progression of the disease as GFR continues to decline, overt proteinuria (i.e., macroalbuminuria), defined as urinary albumin excretion of >300 mg/day ensues and is associated with worsening hypertension. Eventually, ESKD (End Stage Kidney Disease) progresses, leading to the need for renal replacement therapy.

In patients with Type 2 Diabetes Mellitus (T2DM), the clinical progression is variable, primarily due to multiple renal insults, including not only hyperglycemia, but also vascular pathology resulting in ischemic renal injury. However, other common features are likely to contribute to renal injury in patients with T2DM include hyperfiltration at the level of the single nephron, proximal tubular glucotoxicity, and a stimulus for tubular cell growth as a result of enhanced sodium coupled glucose transport into tubular cells.

Studies have demonstrated that albuminuria is a biomarker for predicting progression of diabetic nephropathy and is a cardiovascular (CV) risk factor. When compared with patients with normo-albuminuria and estimated glomerular filtration rate (eGFR) ≥90 mL/min/1.73m², patients with both macroalbuminuria and eGFR <60 mL/min/1.73m² were at 5.9-fold higher risk (95% Cl 3.5 to 10.2) for cardiovascular death and 22.2-fold higher risk (95% Cl 7.6 to 64.7) for experiencing ESKD, and subjects with macroalbuminuria and reduced eGFR (ie, <60 mL/min/1.73m²) were nearly 6 times more likely to experience a composite renal event (i.e., death as a result of kidney disease, requirement for dialysis or transplantation, or doubling of serum creatinine. See, e.g., J Am Soc Nephrol 20(8):1813-1821, 2009. A close link between the degree of albuminuria and CV disease has also been demonstrated in the RENAAL study, showing that patients with high baseline urinary albumin/creatinine ratio (ACR) (≥3 g/g) had a 1.2-fold (95% Cl, 1.54 to 2.38) higher risk of a composite of myocardial infarction (MI), stroke, first hospitalization for heart failure or unstable angina, coronary or peripheral revascularization, or CV death, and a 2.7-fold (95% Cl, 1.94 to 3.75) higher risk of heart failure compared with patients with an ACR <1.5 g/g. Increased urinary albumin excretion and reduced eGFR are also independently associated with the risk for both cardiovascular and kidney outcomes in patients with T2DM, without evidence for an interaction between these risk factors. Moderately increased albuminuria also has been associated with an increase in renal disease progression.

In summary, the magnitude of albuminuria positively correlates with the development of ESKD and adverse CV outcomes. Treatment-related reductions in albuminuria in patients with T2DM and albuminuria using agents acting by a hemodynamic mechanism (i.e., ACEi and ARBs) are correlated with reductions in the progression of diabetic nephropathy and in the incidence of adverse CV outcomes. Thus, agents acting by a unique hemodynamic mechanism to reduce albuminuria beyond that seen with other antihypertensive or antihyperglycemic agents and which are additive to agents disrupting the renin-angiotensin system may exert reno-protective effects and possibly reduce adverse CV outcomes in diabetic nephropathy.

Fatty liver, also known as fatty liver disease (FLD), is a reversible condition wherein large vacuoles of triglyceride fat accumulate in liver cells via the process of steatosis (i.e., abnormal retention of lipids within a cell). Accumulation of fat may also be accompanied by a progressive inflammation of the liver (hepatitis), called steatohepatitis. By considering the contribution by alcohol, fatty liver may be termed alcoholic steatosis or nonalcoholic fatty liver disease (NAFLD), and the more severe forms as alcoholic steatohepatitis (part of alcoholic liver disease) and Non-alcoholic steatohepatitis (NASH).

Non-alcoholic fatty liver disease (NAFLD) is one cause of a fatty liver, occurring when fat is deposited (steatosis) in the liver. NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. Non-alcoholic steatohepatitis (NASH) is a progressive, severe form of NAFLD. Over a 10-year period, up to 20% of patients with NASH will develop cirrhosis of the liver, and 10% will suffer death related to liver disease. The exact cause of NAFLD is still unknown, however, both obesity and insulin resistance are thought to play a strong role in the disease process. The exact reasons and mechanisms by which the disease progresses from one stage to the next are not known.

NAFLD has been linked to insulin resistance (IR) and the metabolic syndrome (MS). As the renin-angiotensin system (RAS) plays a central role in insulin resistance, and subsequently in NAFLD and NASH, an attempt to block the deleterious effects of RAS overexpression has been proposed a target for treatment. While many potential therapies tested in NASH target only the consequences of this condition, or try to “get rid” of excessive fat, angiotensin receptor blockers (ARBs) may act as a tool for correction of the various imbalances that act in harmony in NASH/NAFLD. Indeed, by inhibiting RAS the intracellular insulin signaling pathway may be improved, resulting in better control of adipose tissue proliferation and adipokine production, as well as more balanced local and systemic levels of various cytokines. At the same time, by controlling the local RAS in the liver fibrosis may be prevented and the cycle that links steatosis to necroinflammation slowed down. (GEORGESCU, E. F., in Advances in Therapy, 2008, pp 1141-1174, Vol. 25, Issue 11)

There remains a need for pharmaceutical therapies for treating, delaying, slowing the progression of and/or preventing renal disorders.

There remains a need for pharmaceutical therapies for treating, delaying, slowing the progression of and/or preventing fatty liver disorder, including, for example, NAFLD and NASH.

### SUMMARY OF THE INVENTION

The present invention is directed to methods for treating, delaying, slowing the progression of and/or preventing renal disorders comprising administering to a subject in need thereof a therapeutically effective amount of co-therapy comprising, consisting of or consisting essentially of (a) canagliflozin and (b) one or more ACE inhibitor(s) or one or more ARB(s).

The present invention is further directed to methods for (a) treating, delaying, slowing the progression of, inducing remission of or preventing microalbuminuria (elevated urine albumin levels); (b) treating, delaying, slowing the progression of, or preventing macroalbuminuria; (c) decreasing urine albumin levels; and/or (d) decreasing albumin/creatinine ratio (ACR); comprising administering to subject in need thereof a therapeutically effective amount of co-therapy comprising, consisting of or consisting essentially of a combination of (a) canagliflozin and (b) one or more ACE inhibitor(s) or one or more ARB(s).

The present invention is further directed to methods for decreasing urine albumin levels by greater than or equal to about 30%, preferably by greater than or equal to about 50%, comprising administering to a subject in need thereof, co-therapy comprising, consisting of or consisting essentially of a therapeutically effective amount of a combination of (a) canagliflozin and (b) one or more ACE inhibitor(s) or one or more ARB(s).

The present invention is further directed to methods for decreasing urine albumin levels in a range of from about 30% to about 90%, preferably in a range of from about 30% to about 70%, more preferably in a range of from about 30% to about 50%, comprising administering to a subject in need thereof, co-therapy comprising, consisting of or consisting essentially of a therapeutically effective amount of a combination of (a) canagliflozin and (b) one or more ACE inhibitor(s) or one or more ARB(s).

The present invention is further directed to methods for decreasing the urine albumin/creatinine ratio by greater than or equal to about 30%, preferably by greater than or equal to about 50%, preferably by greater than or equal to about 80%, comprising administering to a subject in need thereof, co-therapy comprising, consisting of or consisting essentially of a therapeutically effective amount of a combination of (a) canagliflozin and (b) one or more ACE inhibitor(s) or one or more ARB(s).

The present invention is further directed to methods for decreasing urine albumin/creatinine ratio in a range of from about 30% to about 90%, preferably in a range of from about 30% to about 70%, more preferably in a range of from about 30% to about 50%, comprising administering to a subject in need thereof, co-therapy comprising, consisting of or consisting essentially of a therapeutically effective amount of a combination of (a) canagliflozin and (b) one or more ACE inhibitor(s) or one or more ARB(s).

The present invention is further directed to methods for preventing, slowing the progression of, delaying and/or treating renal hyperfiltrative injury comprising administering to a subject in need thereof, co-therapy comprising, consisting of or consisting essentially of a therapeutically effective amount of a combination of (a) canagliflozin and (b) one or more ACE inhibitor(s) or one or more AR B(s).

The present invention is further directed to methods for preventing, slowing the progression of, delaying or treating a condition or disorder selected from the group consisting of hyperfiltrative diabetic nephropathy, renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, and obesity comprising administering to a subject in need thereof, co-therapy comprising, consisting of or consisting essentially of a therapeutically effective amount of a combination of (a) canagliflozin and (b) one or more ACE inhibitor(s) or one or more ARB(s).

The present invention is further directed to methods for preventing, slowing the progression of, delaying or treating diabetic nephropathy, comprising administering to a subject in need thereof, a therapeutically effective amount of co-therapy comprising, consisting of or consisting essentially of (a) canagliflozin and (b) one or more ACE inhibitor(s) or one or more ARB(s).

The present invention is further directed to methods for preventing, slowing the progression of or delaying the need for renal replacement therapy (including kidney dialysis, kidney transplant, etc.) in a subject with diabetic nephropathy, comprising administering to the subject a therapeutically effective amount of co-therapy comprising, consisting of or consisting essentially of (a) canagliflozin and (b) one or more ACE inhibitor(s) or one or more ARB(s).

The present invention is further directed to methods for preventing, slowing the progression of or delaying renal death in a subject with diabetic nephropathy, comprising administering to the subject a therapeutically effective amount of co-therapy comprising, consisting of or consisting essentially of (a) canagliflozin and (b) one or more ACE inhibitor(s) or one or more ARB(s).

The present invention is further directed to methods of preventing the occurrence of a cardiovascular event, in a subject with diabetic nephropathy, comprising administering to the subject a therapeutically effective amount of co-therapy comprising, consisting of or consisting essentially of (a) canagliflozin and (b) one or more ACE inhibitor(s) or one or more ARB(s).

The present invention is further directed to methods for treating, delaying, slowing the progression of and/or preventing fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis) comprising administering to a subject in need thereof a therapeutically effective amount of canagliflozin.

The present invention is further directed to methods for treating, delaying, slowing the progression of and/or preventing fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis), comprising administering to a subject in need thereof a therapeutically effective amount of co-therapy comprising, consisting of or consisting essentially of (a) canagliflozin and (b) one or more ACE inhibitor(s) or one or more ARB(s).

The present invention is further directed to methods for treating, delaying, slowing the progression of and/or preventing fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis), comprising administering to a subject in need thereof a therapeutically effective amount of co-therapy comprising, consisting of or consisting essentially of (a) canagliflozin and (b) one or more PPAR-gamma agonists.

The present invention is further directed to methods for (a) treating, delaying, slowing the progression of or preventing alcoholic simple fatty liver; (b) treating, delaying, slowing the progression of or preventing alcoholic steatohepatitis (ASH) (including alcoholic hepatic fibrosis); (c) treating, delaying, slowing the progression of or preventing alcoholic hepatic fibrosis; (d) treating, delaying, slowing the progression of or preventing alcoholic cirrhosis; (e) treating, delaying, slowing the progression of or preventing NAFLD, (f) treating, delaying, slowing the progression of or preventing nonalcoholic simple fatty liver; (g) treating, delaying, slowing the progression of or preventing NASH; (h) treating, delaying, slowing the progression of or preventing nonalcoholic hepatic fibrosis; and/or (i) treating, delaying, slowing the progression of or preventing nonalcoholic cirrhosis; comprising administering to subject in need thereof a therapeutically effective amount of canagliflozin.

The present invention is further directed to methods for (a) treating, delaying, slowing the progression of or preventing alcoholic simple fatty liver;

(b) treating, delaying, slowing the progression of or preventing alcoholic steatohepatitis (ASH) (including alcoholic hepatic fibrosis); (c) treating, delaying, slowing the progression of or preventing alcoholic hepatic fibrosis; (d) treating, delaying, slowing the progression of or preventing alcoholic cirrhosis; (e) treating, delaying, slowing the progression of or preventing NAFLD, (f) treating, delaying, slowing the progression of or preventing nonalcoholic simple fatty liver; (g) treating, delaying, slowing the progression of or preventing NASH; (h) treating, delaying, slowing the progression of or preventing nonalcoholic hepatic fibrosis; and/or (i) treating, delaying, slowing the progression of or preventing nonalcoholic cirrhosis; comprising administering to subject in need thereof a therapeutically effective amount of co-therapy comprising, consisting of or consisting essentially of a combination of (a) canagliflozin and (b) one or more ACE inhibitor(s) or one or more ARB(s).

The present invention is further directed to methods for (a) treating, delaying, slowing the progression of or preventing alcoholic simple fatty liver; (b) treating, delaying, slowing the progression of or preventing alcoholic steatohepatitis (ASH) (including alcoholic hepatic fibrosis); (c) treating, delaying, slowing the progression of or preventing alcoholic hepatic fibrosis; (d) treating, delaying, slowing the progression of or preventing alcoholic cirrhosis; (e) treating, delaying, slowing the progression of or preventing NAFLD, (f) treating, delaying, slowing the progression of or preventing nonalcoholic simple fatty liver; (g) treating, delaying, slowing the progression of or preventing NASH; (h) treating, delaying, slowing the progression of or preventing nonalcoholic hepatic fibrosis; and/or (i) treating, delaying, slowing the progression of or preventing nonalcoholic cirrhosis; comprising administering to subject in need thereof a therapeutically effective amount of co-therapy comprising, consisting of or consisting essentially of a combination of (a) canagliflozin and (b) one or more PPAR-gamma agonists.

In a further embodiment, the present invention is directed to a pharmaceutical composition comprising (a) canagliflozin, (b) one or more ACE inhibitor(s) or one or more ARB(s) and (c) a pharmaceutically acceptable carrier. An illustration of the invention is a pharmaceutical composition made by mixing (a) canagliflozin, (b) one or more ACE inhibitor(s) or one or more ARB(s) and (c) a pharmaceutically acceptable carrier. In a further embodiment the invention is further directed to a process for making a pharmaceutical composition comprising mixing (a) canagliflozin, (b) one or more ACE inhibitor(s) or one or more ARB(s) and (c) a pharmaceutically acceptable carrier.

In a further embodiment, the present invention is directed to a pharmaceutical composition comprising (a) canagliflozin, (b) one or more PPAR-gamma agonists and (c) a pharmaceutically acceptable carrier. An illustration of the invention is a pharmaceutical composition made by mixing (a) canagliflozin, (b) one or more PPAR-gamma agonists and (c) a pharmaceutically acceptable carrier. In a further embodiment the invention is further directed to a process for making a pharmaceutical composition comprising mixing (a) canagliflozin, (b) one or more PPAR-gamma agonists and (c) a pharmaceutically acceptable carrier.

In certain embodiments the invention is directed to a method of treating renal disorders (selected from the group consisting of elevated urine albumin level, elevated albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity) comprising administering to a subject in need thereof a therapeutically effective amount of co-therapy comprising, consisting of or consisting essentially of a combination of (a) canagliflozin and (b) one or more ACE inhibitor(s) or one or more ARB(s),or a pharmaceutical composition as described above.

In an embodiment, the present invention is directed to canagliflozin in combination with one or more ACE inhibitor(s) or one or more ARB(s) for use as a medicament. In another embodiment, the present invention is directed to canagliflozin in combination with one or more ACE inhibitor(s) or one or more ARB(s) for use in the treatment of renal disorders (such as elevated urine albumin level, elevated albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity). In another embodiment, the present invention is directed to a composition comprising canagliflozin and one or more ACE inhibitor(s) or one or more ARB(s) for the treatment of renal disorders (such as elevated urine albumin level, elevated albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity).

Another example of the invention is the use of canagliflozin in combination with one or more ACE inhibitor(s) or one or more ARB(s) in the preparation of a medicament for treating: (a) elevated urine albumin level, (b) elevated serum albumin/creatinine ratio, (c) microalbuminuria, (d) macroalbuminuria, (e) renal hyperfiltrative injury, (f) diabetic nephropathy (including, but not limited to hyperfiltrative diabetic nephropathy), (g) renal hyperfiltration, (h) glomerular hyperfiltration, (i) renal allograft hyperfiltration, (j) compensatory hyperfiltration, (k) hyperfiltrative chronic kidney disease, (l) hyperfiltrative acute renal failure or (m) obesity; in a subject in need thereof.

In another example, the present invention is directed to canagliflozin in combination with one or more ACE inhibitor(s) or one or more ARB(s) in a method for treating renal disorders (such as elevated urine albumin level, elevated serum albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity) in a subject in need thereof.

In certain embodiments the invention is directed to a method of treating fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH), comprising administering to a subject in need thereof a therapeutically effective amount of canagliflozin or a pharmaceutical composition comprising canagliflozin.

In certain embodiments the invention is directed to a method of treating fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH) comprising administering to a subject in need thereof a therapeutically effective amount of co-therapy comprising, consisting of or consisting essentially of a combination of (a) canagliflozin and (b) one or more ACE inhibitor(s) or one or more ARB(s) or a pharmaceutical composition as described above.

In certain embodiments the invention is directed to a method of treating fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH) comprising administering to a subject in need thereof a therapeutically effective amount of co-therapy comprising, consisting of or consisting essentially of a combination of (a) canagliflozin and (b) one or more PPAR-gamma agonists or a pharmaceutical composition as described above.

In another embodiment, the present invention is directed to canagliflozin for use in the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH). In another embodiment, the present invention is directed to a composition comprising canagliflozin for the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH).

In another embodiment, the present invention is directed to canagliflozin in combination with one or more ACE inhibitor(s) or one or more ARB(s) for use in the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH). In another embodiment, the present invention is directed to a composition comprising canagliflozin and one or more ACE inhibitor(s) or one or more ARB(s) for the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH) .

In another embodiment, the present invention is directed to canagliflozin in combination with one or more PPAR-gamma agonists for use in the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH). In another embodiment, the present invention is directed to a composition comprising canagliflozin and one or more PPAR-gamma agonists for the treatment of fatty liver disorders (including, but not limited to, alcoholic simple fatty liver, alcoholic steatohepatitis (ASH) (including alcoholic hepatic fibrosis), alcoholic hepatic fibrosis, alcoholic cirrhosis, nonalcoholic fatty liver disease (NAFLD), nonalcoholic simple fatty liver, nonalcoholic steatohepatitis (NASH), nonalcoholic hepatic fibrosis and nonalcoholic cirrhosis; preferably NAFLD or NASH).

Another example of the invention is the use of canagliflozin in the preparation of a medicament for treating: (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis; in a subject in need thereof. In another example, the present invention is directed to canagliflozin in a methods for (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis; in a subject in need thereof.

Another example of the invention is the use of canagliflozin in combination with one or more ACE inhibitor(s) or one or more ARB(s) in the preparation of a medicament for treating: (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis; in a subject in need thereof. In another example, the present invention is directed to canagliflozin in combination with one or more ACE inhibitor(s) and/or one or more ARB(s) in a methods for treating (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis; in a subject in need thereof.

Another example of the invention is the use of canagliflozin in combination with one or more PPAR-gamma agonists in the preparation of a medicament for treating: (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis in a subject in need thereof. In another example, the present invention is directed to canagliflozin in combination with one or more PPAR-gamma agonists in a methods for treating (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; or (i) nonalcoholic cirrhosis; in a subject in need thereof.

### BRIEF DESCRIPTION OF THE FIGURES

FIG. 1 illustrates the median % change from baseline over time in albumin/creatinine ratio in the CANVAS clinical trial, in subjects with microalbuminuria.

FIG. 2 illustrates the median % change from baseline over time in albumin/creatinine ratio in the CANVAS clinical trial, in subjects with macroalbuminuria.

FIG. 3 illustrates eGFR (mL/min/1.73m2) mean change from baseline over time, regardless of rescue medication, within 2 days of last study medication in the CANVAS clinical trial.

FIG. 4 illustrates eGFR (mL/min/1.73m2) mean change from baseline over time in the DIA3004 clinical trial.

FIG. 5 illustrates eGFR (mL/min/1.73m2) mean change from baseline over time, regardless of rescue medication, within 2 days of last study medication in the DIA3009 clinical trial.

### DETAILED DESCRIPTION OF THE INVENTION

The present invention is directed to methods for preventing, slowing the progression of, delaying and/or treating renal disorders, comprising administering to a subject in need thereof a therapeutically effective amount of co-therapy; wherein the co-therapy comprises, consists of or consists essentially of (a) canagliflozin and (b) one or more ACE inhibitor(s) or one or more ARB(s).

The present invention is further directed to methods for preventing, slowing the progression of, delaying and/or treating a fatty liver disorder selected from the group consisting of (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; and (i) nonalcoholic cirrhosis; comprising administering to a subject in need thereof a therapeutically effective amount of canagliflozin.

The present invention is further directed to methods for preventing, slowing the progression of, delaying and/or treating a fatty liver disorder selected from the group consisting of of (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; and (i) nonalcoholic cirrhosis; comprising administering to a subject in need thereof a therapeutically effective amount of co-therapy; wherein the co-therapy comprises, consists of or consists essentially of (a) canagliflozin and (b) one or more ACE inhibitor(s) or one or more ARB(s).

The present invention is further directed to methods for preventing, slowing the progression of, delaying and/or treating a fatty liver disorder selected from the group consisting of (a) alcoholic simple fatty liver, (b) alcoholic steatohepatitis (ASH) (including alcoholic hepatic fibrosis), (c) alcoholic hepatic fibrosis, (d) alcoholic cirrhosis, (e) nonalcoholic fatty liver disease (NAFLD), (f) nonalcoholic simple fatty liver, (g) nonalcoholic steatohepatitis (NASH), (h) nonalcoholic hepatic fibrosis; and (i) nonalcoholic cirrhosis; comprising administering to a subject in need thereof a therapeutically effective amount of co-therapy; wherein the co-therapy comprises, consists of or consists essentially of (a) canagliflozin and (b) one or more PPAR-gamma agonists.

In an embodiment or the present invention, the subject in need thereof is any individual diagnosed or showing one or more symptoms of any of the following:

(a) diabetes mellitus (regardless of type);

(b) chronic kidney disease (CKD);

(c) acute renal failure (ARF);

(d) renal transplant recipients;

(e) renal transplant donors;

(f) unilateral total or partial nephrectomized patients; or

(g) nephrotic syndrome.

In a preferred embodiment of the present invention, the subject in need thereof has been diagnosed with or shows symptoms of diabetes mellitus. In another embodiment, the subject in need thereof has been diagnosed with or shows symptoms of Type 1 diabetes mellitus or Type 2 diabetes mellitus. In another embodiment, the subject in need thereof has been diagnosed with or shows symptoms of Type 1 diabetes mellitus. In another embodiment, the subject in need thereof has been diagnosed with or shows symptoms of Type 2 diabetes mellitus. In another embodiment of the present invention, the subject in need thereof has been diagnosed with or shows symptoms of Type 2 diabetes mellitus and insufficient glycemic control. In another embodiment of the present invention, the subject in need thereof has been diagnosed with or shows symptoms of Type 2 diabetes mellitus and diabetic nephropathy.

In another embodiment of the present invention, the subject in need thereof is any individual diagnosed with or showing symptoms of other types of diabetes mellitus, such as for example, maturity onset diabetes of the youth (MODY), latent autoimmune diabetes of adults (LADA) or pre-diabetes. In another embodiment of the present invention, the subject in need thereof is any individual diagnosed with or showing symptoms of pre-diabetes, elevated blood glucose levels or impaired glucose tolerance. In another embodiment of the present invention, the subject in need thereof is any individual diagnosed with or showing symptoms of metabolic syndrome (also called Syndrome X).

In an embodiment of the present invention, the subject in need thereof is a patient whose measured GFR is equal to or greater than about 125 mL/min/1.73 m². In another embodiment of the present invention, the subject in need thereof is a patient whose measured GFR is equal to or greater than about 140 mL/min/1.73 m².

In another embodiment of the present invention, the subject in need thereof is:
(1) an individual diagnosed of one or more of the conditions selected from the group consisting of overweight, obesity, visceral obesity and abdominal obesity; or
(2) an individual who exhibits one, two or more of the following signs:
   (a) a fasting blood glucose or serum glucose concentration greater than about 100 mg/dL, preferably, greater than about 125 mg/dL;
   (b) a postprandial plasma glucose equal to or greater than about 140 mg/dL;
   (c) an HbA1c value equal to or greater than about 6.0%, preferably equal to or greater than about 6.5%, preferably equal to or greater than 7.0%, preferably equal to or greater than about 7.5%, preferably equal to or greater than about 8.5%; or
(3) an individual in whom one, two, three or more of the following conditions are present:
   (a) obesity, visceral obesity and/or abdominal obesity,
   (b) triglyceride blood level equal to or greater than about 150 mg/dL,
   (c) HDL-cholesterol blood level less than about 40 mg/dL in female patients and less than about 50 mg/dL in male patients,
   (d) a systolic blood pressure equal to or greater than about 130 mm Hg and a diastolic blood pressure equal to or greater than about 85 mm Hg,
   (e) a fasting blood glucose level equal to or greater than about 100 mg/dL; or
(4) an individual with obesity (an individual with a calculated BMI of greater than about 30, more preferably an individual with a calculated BMI of greater than about 35), more preferably an individual with morbidly obesity (an individual with a calculated BMI of greater than about 40 or a calculated BMI of greater than about 35 and a comorbidity such as diabetes mellitus or hypertension).

In an embodiment or the present invention, the subject in need thereof is any individual diagnosed or showing one or more symptoms of any of the following:

(a) alcoholic simple fatty liver;

(b) alcoholic steatohepatitis (ASH) (including alcoholic hepatic fibrosis);

(c) alcoholic hepatic fibrosis;

(d) alcoholic cirrhosis;

(e) nonalcoholic fatty liver disease (NAFLD);

(f) nonalcoholic simple fatty liver;

(g) nonalcoholic steatohepatitis (NASH);

(h) nonalcoholic hepatic fibrosis; or

(i) nonalcoholic cirrhosis.

In another embodiment or the present invention, the subject in need thereof is any individual diagnosed or showing one or more symptoms of any of the following: (a) nonalcoholic fatty liver disease (NAFLD); (b) nonalcoholic simple fatty liver; (c) nonalcoholic steatohepatitis (NASH); (d) nonalcoholic hepatic fibrosis; or (e) nonalcoholic cirrhosis. In another embodiment or the present invention, the subject in need thereof is any individual diagnosed or showing one or more symptoms of any of the following: (a) NAFLD or (b) NASH.

### Definitions

As used herein, unless otherwise noted, the term “canagliflozin” shall mean a compound of formula (I-X)

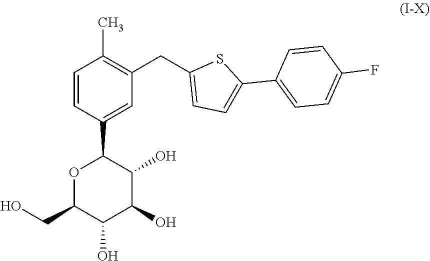 [SMILES: Cc1ccc([C@@H]2O[C@H](CO)[C@@H](O)[C@H](O)[C@H]2O)cc1Cc1ccc(-c2ccc(F)cc2)s1]

or a crystalline hemihydrate form of the compound of formula (I-X). The compound of formula (I-X) exhibits inhibitory activity against sodium-dependent glucose transporter, such as for example SGLT2; and may be prepared according to the process as disclosed in Nomura, S. et al., US Patent Publication, US 2005/0233988 A1, published Oct. 20, 2005, which is incorporated by reference herein.

As used herein, the term “canagliflozin” shall further include a mixture of stereoisomers, or each pure or substantially pure isomer. In addition, the term “canagliflozin” shall include an intramolecular salt, hydrate, solvate or polymorph thereof. In an embodiment, the term “canagliflozin” shall mean the crystalline hemihydrate form of the compound of formula (I-X), as described in WO 2008/069327, the disclosure of which is hereby incorporated by reference in its entirety.

In an embodiment of the present invention, canagliflozin is administered in an amount in the range of from about 50 to about 500 mg. In another embodiment of the present invention, canagliflozin is in an amount in the range of from about 100 to about 300 mg. In another embodiment of the present invention, canagliflozin is administered in an amount of about 100 mg. In another embodiment of the present invention, canagliflozin is administered in an amount of about 300 mg.

As used herein, unless otherwise noted, the term “ACE inhibitor” or “angiotensin-converting-enzyme inhibitor” shall mean any pharmaceutical agent which inhibits the angiotensin-converting enzyme, thereby decreasing the tension of blood vessels and blood volume (i.e. dilating blood vessels), thus lowering blood pressure. As such ACE inhibitors may be used in the treatment of hypertension, acute myocardial infarction (MI, heart attack), cardiac failure (e.g. left ventricular systolic dysfunction), congestive heart failure, renal complication of diabetes mellitus (e.g. diabetic nephropathy), chronic renal failure and renal involvement in systemic sclerosis.

ACE inhibitors can be divided into three groups based on their molecular structure: (a) Sulfhydryl-containing agents including, but not limited to, alacepril, captopril (CAPOTEN®) and zofenopril; (b) Dicarboxylate-containing agents including, but not limited to, enalapril (VASOTEC®), ramipril (ALTACE®, PRILACE®, RAMACE®), quinapril (ACCUPRIL®), perindopril (COVERSYL®, ACEON®), lisinopril (PRINIVIL®, ZESTRIL®), benazepril (LOTENSIN®), imidapril (TANATRIL®, TANAPRESS®, CARDIPRIL®), zofenopril (ZOFECARD®), trandolapril (MAVIK®, ODRIK®), moexipril (UNIVASC®), cilazapril, delapril, spirapril, and temocapril; and (c) Phosphonate-containing agents including, but not limited to, fosinopril (FOSITEN®, MONOPRIL®). Preferably, the ACE inhibitor is selected from the group consisting of benazepril, captopril, enalapril, imidapril, lisinopril and ramipril. More preferably, the ACE inhibitor is selected from the group consisting of enalapril, imidapril, lisinopril and ramipril.

In an embodiment of the present invention, the ACE inhibitor is selected from the group consisting of benazepril, captopril, enalapril, imidapril, lisinopril and ramipril. In another embodiment of the present invention, the ACE inhibitor is selected from the group consisting of enalapril, imidapril, lisinopril and ramipril.

As used herein, unless otherwise noted, the term “ARB” and “antigiotesin receptor blockers” and “angiotensin II receptor antagonists” shall mean any pharmaceutical agent which modulates the renin-angiotensin-aldosterone system. More particularly, ARBs block activation of angiotensin II AT1 receptors, which results in vasodilation (dilation of blood vessels), reduced secretion of vasopressin and reduced production and secretion of aldosterone, among other actions. The combined effect reduces blood pressure. As such ARBs may be used in the treatment of hypertension, diabetic nephropathy and congestive heart failure.

Suitable examples of ARBs include, but are not limited to, losartan (COZAAR®), irbesartan (APROVEL®, KARVEA®, AVAPRO®), olmesartan (BENICAR®), candesartan (BLOPRESS®, ATACAND®), valsartan (DIOVAN®), telmisartan (MICARDIS®), azilsartan (EDARBI®) and eprosartan (TEVETAN®). Preferably, the ARB is selected from the group consisting of candesartan, irbesartan, losartan and valsartan. More preferably, the ARB is selected from the group consisting of irbesartan and losartan.

In an embodiment of the present invention, the ARB is selected from the group consisting of candesartan, irbesartan, losartan and valsartan. In another embodiment of the present invention, ARB is selected from the group consisting of irbesartan and losartan.

As used herein, unless otherwise noted, the term “PPAR-gamma agonist” shall mean any pharmaceutical agent which acts as an agonist of the peroxisome proliferator-activated receptor gamma (PPAR-gamma), useful in lowering blood sugar, lowering triglycerides, and the like. Suitable example include thiazolidinediones (TZDs), used in the treatment of for example, Type 2 diabetes mellitus and other disorders exhibiting insulin resistance.

Suitably examples of PPAR-gamma agonists include, but are not limited to pioglitazone (ACTOS®), rivoglitazone, rosiglitazone (AVANDIA®), troglitazone, netoglitazone, ciglitazone, and the like. Preferably, the PPAR-gamma agonist is selected from the group consisting of pioglitazone, rosiglitazone and troglitazone. More preferably, the PPAR-gamma agonist is selected from the group consisting of pioglitazone and rosiglitazone.

In an embodiment of the present invention, the PPAR-gamma agonist is selected from the group consisting of pioglitazone, rivoglitazone, rosiglitazone, troglitazone, netoglitazone and ciglitazone. In another embodiment of the present invention, the PPAR-gamma agonist is selected from the group consisting of pioglitazone, rosiglitazone and troglitazone.

One skilled in the art will readily recognize that recommended dosage amounts and regimens for known and/or marketed ACE Inhibitors, ARBs and PPAR-gamma agonists may be determined by consulting appropriate references such as drug package inserts, FDA guidelines, the Physician's Desk Reference, and the like.

As used herein, unless otherwise noted, the term “renal disorders” shall mean any disorder related to or affecting kidney function and/or renal hyperfiltration. Renal disorders including, but are not limited to elevated urine albumin level, elevated serum albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity.

According to the National Kidney Foundation (NKF) Kidney Disease Outcomes Quality Initiative (KDOQI), Guidelines for Screening and Diagnosis of Diabetic Kidney Disease, microalbuminuria is diagnosed in a subject (patient) whose albumin-creatinine ratio (ACR) is between 30 mg/g and 300 mg/g; and macroalbuminuria is diagnosed in a subject (patient) whose albumin-creatinine ration (ACR) is greater than 300 mg/g.

The term “hyperfiltration” is defined as an elevation in the filtration rate of the renal glomeruli. In one aspect, hyperfiltration is defined as a whole kidney filtration rate equal to or greater than about 125 mL/min/1.73 m², especially equal to or greater than about 140 mL/min/1.73 m², as measured using a method described herein below. Hyperfiltration may also be defined as related to an absolute GFR greater to the about 90^{th}, or the about 95^{th}, percentile in the studied population after adjusting for sex, age, weight, height, and the use of ACE inhibitors or ARB (Melsom et al. Diabetes Care 2011; DOI: 10.2337/dc11-0235).

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) may be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR is typically recorded in units of volume per time, e.g., milliliters per minute and the formula below can be used:

$$G F R = \frac{( Urine Concentration \times Urine Volume )}{Plasma Concentration}$$

The GFR may be determined by injecting inulin into the plasma. Since inulin is neither reabsorbed nor secreted by the kidney after glomerular filtration, its rate of excretion is directly proportional to the rate of filtration of water and solutes across the glomerular filter. A normal value is: GFR=90-125 mL/min/1.73 m², in particular GFR=100-125 mL/min/1.73 m². Other principles to determine GFR involve measuring 51Cr-EDTA, [1251]iothalamate or iohexyl.

The “estimated glomerular filtration rate (eGFR)” is defined as derived at screening from serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. Subjects with normal renal function are defined as eGFR equal to or greater than 90 ml/min. Subjects with mild impairment of renal function as defined eGFR equal to or greater than 60 and less than 90 ml/min). Subjects with moderate impairment as defined as eGFR equal to or greater than 30 and less than 60 ml/min). Subjects with severe impairment as defined as eGFR equal to or greater than 15 and less than 30 ml/min.

The term “renal hyperfiltrative injury” is defined as a manifestation of renal damage caused predominantly by renal hyperfiltration, which often is an early link in the chain of events to further renal injury, acknowledging that hyperfiltration often works in concert with other chronic kidney disease risk factors in the pathogenesis of renal injury.

The term “body mass index” or “BMI” of a human patient is defined as the weight in kilograms divided by the square of the height in meters, such that BMI has units of kg/m². The term “overweight” is defined as the condition wherein the adult individual of Europide origin has a BMI greater than or 25 kg/m² and less than 30 kg/m². In subjects of Asian origin the term “overweight” is defined as the condition wherein the adult individual has a BMI greater than or 23 kg/m² and less than 25 kg/m². The terms “overweight” and “pre-obese” are used interchangeably.

The term “obesity” is defined as the condition wherein the adult individual of Europid origin has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the terms “class III obesity” is the condition wherein the BMI is equal to or greater than 40 kg/m². In subjects of Asian origin the term “obesity” is defined as the condition wherein the adult individual has a BMI equal or greater than 25 kg/m². Obesity in Asians may be categorized further as follows: the term “class I obesity” is the condition wherein the BMI is equal to or greater than 25 kg/m²but lower than 30 kg/m²; the term “class II obesity” is the condition wherein the BMI is equal to or greater than 30 kg/m².

The term “visceral obesity” is defined as the condition wherein a waist-to-hip ratio of greater than or equal to 1.0 in men and 0.8 in women is measured. It defines the risk for insulin resistance and the development of pre-diabetes. The term “abdominal obesity” is usually defined as the condition wherein the waist circumference is >40 inches or 102 cm in men, and is >35 inches or 94 cm in women (for normal ranges of populations, see for example “Joint scientific statement (IDF, NHLBI, AHA, WHO, IAS, IASO). Circulation 2009; 120:1640-1645”).

The term “morbid obesity” is defined herein as a condition in which the individual of Europid origin has a BMI >40 or has a BMI >35 and a comorbidity such as diabetes melitus or hypertension (see World Health Organization. Obesity: Preventing and Managing the Global Epidemic: Report on a WHO Consultation. World Health Organ Tech Rep Ser. 2000; 894: i-xii, 1-253).

The term “fasting” has the usual meaning as a medical term.

The term “euglycemia” is defined as the condition in which a subject has a fasting blood glucose concentration within the normal range, greater than 70 mg/dL (3.89 mmol/L) and less than 100 mg/dL (5.6 mmol/L), and a 2 h postprandial glucose concentration less than 140 mg/dl.

The term “hyperglycemia” is defined as the condition in which a subject has a fasting blood glucose concentration above the normal range, greater than 100 mg/dL (5.6 mmol/L).

The term “hypoglycemia” is defined as the condition in which a subject has a blood glucose concentration below the normal range, in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l. A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio<1.0 (for men) or <0.8 (for women).

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or Type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled

“The Prevention or Delay of Type 2 Diabetes” issued jointly by the American Diabetes Association and the National Institute of Diabetes and Digestive and Kidney Diseases (Diabetes Care 2002; 25:742-749). Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

The term “Type 2 diabetes” is defined as the condition in which a subject has a fasting (i.e., no caloric intake for 8 hours) blood glucose or serum glucose concentration greater than 125 mg/dL (6.94 mmol/L), when measured at minimum two independent occasions. The measurement of blood glucose values is a standard procedure in routine medical analysis. Type 2 diabetes is also defined as the condition in which a subject has HbA1c equal to, or greater than 6.5%, a two hour plasma glucose equal to, or greater than 200 mg/dL (11.1 mmol/L) during an oral glucose tolerance test (OGTT) or a random glucose concentration equal to, or greater than 200 mg/dL (11.1 mmol/L) in conjunction with classic symptoms of hyperglycaemia or hyperglycaemic crisis. In the absence of unequicoval hyperglycaemia, as with most diagnostic tests, a test result diagnostic of diabetes should be repeated to rule out laboratory error. The assessment of HbA1c should be performed using a method certified by the National Glycohemoglobin Standardization Program (NGSP) and standardized or traceable to the Diabetes Control and Complications Trial (DCCT) reference assay. If a OGTT is carried out, the blood sugar level of a diabetic will be in excess of 200 mg of glucose per dL (11.1 mmol/I) of plasma 2 hours after 75 g of glucose have been taken on an empty stomach. In a glucose tolerance test 75 g of glucose are administered orally to the patient being tested after a minimum of 8 hours, typically after 10-12 hours, of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage Type 2 diabetes mellitus” includes patients with a long-standing duration of diabetes, secondary drug failure, indication for insulin therapy and potentially progression to micro- and macrovascular complications e.g. diabetic nephropathy, or coronary heart disease (CHD).

The term “Type 1 diabetes” is defined as the condition in which a subject has, in the presence of autoimmunity towards the pancreatic beta-cell (i.e.

detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging), a fasting (i.e., no caloric intake for 8 hours) blood glucose or serum glucose concentration greater than 125 mg/dL (6.94 mmol/L). Type 1 diabetes is also defined as the condition in which a subject has, in the presence of autoimmunity towards the pancreatic beta-cell, HbA1c equal to, or greater than 6.5%, a two hour plasma glucose equal to, or greater than 200 mg/dL (11.1 mmol/L) during an oral glucose tolerance test (OGTT) or a random glucose equal to, or greater than 200 mg/dL (11.1 mmol/L) in conjunction with classic symptoms of hyperglycaemia or hyperglycaemic crisis. In the absence of uneqicoval hyperglycaemia, as with most diagnostic tests, a test result diagnostic of diabetes should be repeated to rule out laboratory error. The measurement of blood glucose values is a standard procedure in routine medical analysis. The assessment of HbA1c should be performed using a method certified by the National Glycohemoglobin Standardization Program (NGSP) and standardized or traceable to the Diabetes Control and Complications Trial (DCCT) reference assay. If an OGTT is carried out, the blood sugar level of a diabetic will be in excess of 200 mg of glucose per dL (11.1 mmol/l) of plasma 2 hours after 75 g of glucose have been taken on an empty stomach, in the presence of autoimmunity towards the pancreatic beta cell. In a glucose tolerance test 75 g of glucose are administered orally to the patient being tested after a minimum of 8 hours, typically, 10-12 hours, of fasting and the blood sugar level is recorded immediately before taking the glucose and 1 and 2 hours after taking it. Typically a genetic predisposition is present (e.g. HLA, INS VNTR and PTPN22), but this is not always the case.

The term “MODY” (“maturity onset diabetes of the youth”) describes a monogenic form for diabetes that, according to gene affects, is split into MODY variants, e.g., MODY 1,2.3.4 etc.

The term “LADA” (“latent autoimmune diabetes of adults”) refers to patients that has a clinical diagnosis of Type 2 Diabetes Mellitus, but who is being detected to have autoimmunity towards the pancreatic beta cell.

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference greater than about 40 inches or 102 cm in men, and greater than about 35 inches or 94 cm in women;
2. Triglycerides equal to or greater than about 150 mg/dL;
3. HDL-cholesterol less than about 40 mg/dL in men and less than about 50 in women;
4. Blood pressure equal to or greater than about 130/85 mm Hg (SBP equal to or greater than about 130 or DBP equal to or greater than about 85);
5. Fasting blood glucose equal to or greater than about 100 mg/dL.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The definitions of NODAT (new onset diabetes after transplantation) and PTMS (post-transplant metabolic syndrome) follow closely that of the American Diabetes Association diagnostic criteria for type 2 diabetes, and that of the International Diabetes Federation (IDF) and the American Heart Association/National Heart, Lung, and Blood Institute, for the metabolic syndrome. NODAT and/or PTMS are associated with an increased risk of micro- and macrovascular disease and events, graft rejection, infection, and death. A number of predictors have been identified as potential risk factors related to NODAT and/or PTMS including a higher age at transplant, male gender, the pre-transplant body mass index, pre-transplant diabetes, and immunosuppression.

The term “gestational diabetes” (diabetes of pregnancy) denotes a form of the diabetes which develops during pregnancy and usually ceases again immediately after the birth. Gestational diabetes is diagnosed by a screening test which often is carried out between the 24th and 28th weeks of pregnancy, but could be conducted at any time during pregnancy, in particular if previous gestational diabetes has been diagnosed. It is usually a simple test in which the blood sugar level is measured e.g., one hour after the administration of 50 g of glucose solution. If this 1 h level is above 140 mg/dl, gestational diabetes is suspected. Final confirmation may be obtained by a standard glucose tolerance test, for example with 75 g of glucose; which also serve as a diagnostic test in the absence of the 50 g challenge.

As used herein, unless otherwise noted, the term “fatty liver disorder” shall mean any disease, disorder or condition characterized by the accumulation of fat (e.g. triglycerides) in the liver cells. Fatty liver disorders include alcoholic liver diseases, disorders and conditions; and nonalcoholic fatty liver diseases, disorders and conditions.

Alcoholic liver disease (also called alcoholic liver injury) is a disease caused by fat accumulation in liver cells as a result of alcohol ingestion. Examples of alcoholic liver disorders include, but are not limited to alcoholic simple fatty liver, alcoholic steatohepatitis (ASH), alcoholic hepatic fibrosis, alcoholic cirrhosis, and the like; wherein alcoholic steatohepatitis is also called alcoholic fatty hepatitis and includes alcoholic hepatic fibrosis.

Nonalcoholic fatty liver disease is a disease with fat deposition in the liver, which occurs in patients whose alcohol ingestion is not enough to cause liver injury, except for cases of known etiology, such as viral hepatitis and autoimmune hepatitis. Examples of nonalcoholic liver disorders include, but are not limited to, nonalcoholic simple fatty liver, nonalcoholic steatohepatitis (NASH), nonalcoholic hepatic fibrosis, nonalcoholic cirrhosis, and the like. Nonalcoholic simple fatty liver is a disease only with fat deposition in liver cells. Nonalcoholic steatohepatitis (NASH) is a disease with liver fatty change, along with inflammation, liver cell necrosis, ballooning and fibrosis, similarly to alcoholic steatohepatitis, and also including nonalcoholic hepatic fibrosis. Nonalcoholic hepatic fibrosis is a disease with advanced fibrosis in liver tissues, along with excessive production and accumulation of collagen and other extracellular matrix components. Nonalcoholic cirrhosis is a disease with reconstructed hepatic lobule structure as a result of advanced fibrosis.

In an embodiment of the present invention, the fatty liver disorder is selected from the group consisting of alcoholic fatty liver disorders, diseases and conditions. In another embodiment of the present invention, the fatty liver disorder is selected from the group consisting of alcoholic simple fatty liver, alcoholic steatohepatitis (ASH), alcoholic hepatic fibrosis, alcoholic cirrhosis, and the like.

In an embodiment of the present invention, the fatty liver disorder is selected from the group consisting of non-alcoholic fatty liver disorders, diseases and conditions. In another embodiment of the present invention, the fatty liver disorder is selected from the group consisting of nonalcoholic simple fatty liver, nonalcoholic steatohepatitis (NASH), nonalcoholic hepatic fibrosis and nonalcoholic cirrhosis. In another embodiment of the present invention, the fatty liver disorder is selected from the group consisting of NAFLD and NASH.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

As used herein, unless otherwise noted, the terms “treating”, “treatment” and the like, shall include the management and care of a subject or patient (preferably a mammal, more preferably a human) for the purpose of combating a disease, condition, or disorder. The terms “treating” and “treatment” include the administration of the compound(s) or pharmaceutical composition(s) as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

As used herein, unless otherwise noted, the terms “delaying the progression of” and “slowing the progression of” shall include (a) delaying or slowing the development of one or more symptoms or complications of the disease, condition or disorder; (b) delaying or slowing the development of one or more new/additional symptoms or complications of the disease, condition or disorder; and/or (c) delaying or slowing the progression of the disease, condition or disorder to a later stage or more serious form of said disease, condition or disorder.

As used herein, unless otherwise noted, the terms “preventing” and “prevention” shall include (a) reducing the frequency of one or more symptoms; (b) reducing the severity of one or more symptoms; (c) delaying, slowing or avoiding of the development of one or more additional symptoms; and/or (d) delaying, slowing or avoiding the development of the disorder, condition or disease to a later stage or more serious form.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to co-therapy or combination therapy, comprising administration of (a) canagliflozin and (b) one or more ACE inhibitor or one or more ARB or one or more PPAR-gamma agonist, “therapeutically effective amount” shall mean that amount of the combination of agents taken together so that the combined effect elicits the desired biological or medicinal response. For example, the therapeutically effective amount of co-therapy comprising administration of (a) canagliflozin and (b) an ACE inhibitor, would be the amount of (a) canagliflozin and (b) the ACE inhibitor that when taken together or sequentially have a combined effect that is therapeutically effective. Further, it will be recognized by one skilled in the art that in the case of co-therapy with a therapeutically effective amount, as in the example above, the amount of the (a) canagliflozin and/or the amount of the (b) ACE inhibitor individually may or may not be therapeutically effective.

Optimal dosages (for canagliflozin, ACE inhibitor, ARB, PPAR-gamma agonist, or co-therapy comprising canagliflozin and one or more ACE inhibitor or one or more ARB or one or more PPAR-gamma agonist) to be administered may be readily determined by those skilled in the art, and will vary with for example, the mode of administration, the strength of the preparation, and the advancement of the disease condition. In addition, factors associated with the particular patient being treated, including patient age, weight, diet and time of administration, will result in the need to adjust dosages.

As used herein, the term “composition” is intended to encompass a product comprising the specified ingredients in the specified amounts, as well as any product which results, directly or indirectly, from combinations of the specified ingredients in the specified amounts.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. Further, to provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Renal filtration and reuptake of glucose contribute, among other mechanisms, to the steady state plasma glucose concentration and can therefore serve as an antidiabetic target. Reuptake of filtered glucose across epithelial cells of the kidney proceeds via sodium-dependent glucose cotransporters (SGLTs) located in the brush-border membranes in the tubuli along the sodium gradient. There are at least 3 SGLT isoforms that differ in their expression pattern as well as in their physico-chemical properties. SGLT2 is almost exclusively expressed in the kidney, whereas SGLT1 is expressed additionally in other tissues like intestine, colon, skeletal and cardiac muscle. SGLT3 has been found to be a glucose sensor in interstitial cells of the intestine without any transport function. Potentially, other related, but not yet characterized genes, may contribute further to renal glucose reuptake. Under normoglycemia, glucose is completely reabsorbed by SGLTs in the kidney, whereas the reuptake capacity of the kidney is saturated at glucose concentrations higher than 10 mM, resulting in glucosuria (“diabetes mellitus”). This threshold concentration can be decreased by SGLT2-inhibition. It has been shown in experiments with the SGLT inhibitor phlorizin that SGLT-inhibition will partially inhibit the reuptake of glucose from the glomerular filtrate into the blood leading to a decrease in blood glucose concentrations and to glucosuria.

In an embodiment, a subject in the context of the present invention is an individual showing renal hyperfiltration or at risk of developing renal hyperfiltration. Such a subject is for example an individual diagnosed or showing diabetes mellitus (see for example Melsom et al. Diabetes Care 2011; DOI: 10.2337/dc11-0235). Such a subject is for example an individual diagnosed or showing Type 1 diabetes mellitus, Type 2 diabetes mellitus, MODY, LADA, pre-diabetes, obesity, congenital or acquired obstructive uro/nephropathy, chronic kidney disease (CKD) and/or acute renal failure (ARF). Such patient is also for example a renal transplant recipient, a renal transplant donor, or an unilateral total or partial nephrectomized patient.

In another embodiment, a subject in the context of the present invention is an individual having glomerular filtration rate (GFR) equal to or above 125 ml/min/1.73 m². In a further aspect, a subject in the context of the present invention is an individual having a GFR equal to or above 140 ml/min/1.73 m². The GFR of the individual is measured by a method known in the art or as described herein.

In an embodiment, the subject is an individual diagnosed with type 1 diabetes mellitus. In another embodiment, the subject is an individual diagnosed with Type 2 diabetes mellitus, MODY, LADA or pre-diabetes. In an embodiment, the subject:
(1) is an individual diagnosed of one or more of the conditions selected from the group consisting of overweight, obesity, visceral obesity and abdominal obesity; or
(2) is an individual who shows one, two or more of the following signs:
   (a) a fasting blood glucose or serum glucose concentration greater than 100 mg/dL, in particular greater than 125 mg/dL;
   (b) a postprandial plasma glucose equal to or greater than 140 mg/dL;
   (c) an HbA1c value equal to or greater than 6.0%, in particular equal to or greater than 6.5%, in particular equal to or greater than 8.0%;
(3) is an individual in whom one, two, three or more of the following conditions are present:
   (a) obesity, visceral obesity and/or abdominal obesity,
   (b) triglyceride blood level ≥150 mg/dL,
   (c) HDL-cholesterol blood level <40 mg/dL in female patients and <50 mg/dL in male patients,
   (d) a systolic blood pressure ≥130 mm Hg and a diastolic blood pressure ≥85 mm Hg,
   (e) a fasting blood glucose level ≥100 mg/dL; or
(4) is an individual with obesity (preferably morbid obesity).

By the administration of the pharmaceutical composition according certain embodiments of the invention and in particular in view of the SGLT2 inhibitory activity of canagliflozin, excessive blood glucose is excreted through the urine of the patient, so that no gain in weight or even a reduction in body weight may result. Therefore, a treatment or prophylaxis according to this invention is advantageously suitable in those patients in need of such treatment or prophylaxis who are diagnosed of one or more of the conditions selected from the group consisting of overweight and obesity, in particular class I obesity, class II obesity, class III obesity, morbid obesity, visceral obesity and abdominal obesity. In addition a treatment or prophylaxis according to this invention is advantageously suitable in those subjects in which a weight increase should preferably be avoided.

Furthermore, the method and/or use according to this invention is advantageously applicable in those subjects who show one, two or more of the following signs:
(a) a fasting blood glucose or serum glucose concentration greater than 100 mg/dL, in particular greater than 125 mg/dL;
(b) a postprandial plasma glucose equal to or greater than 140 mg/dL;
(c) an HbA1c value equal to or greater than 6.0%, equal to or greater than 6.5%, equal to or greater than 7.0%, equal to or greater than 7.5%, or equal to or greater than 8.0%.

The methods and uses according to the present invention may be of particularly advantageous in those subjects who are pre-treated with an antidiabetic medicament and who have a risk to develop hyperfiltration or who are diagnosed of having hyperfiltration. The methods and uses according to the present invention may also be of particularly advantageous in those subjects who are pre-treated with an antidiabetic medicament and who have a risk to develop diabetic nephropathy or who are diagnosed of having diabetic nephropathy.

20

The present invention further comprises pharmaceutical compositions containing canagliflozin and one or more pharmaceutically acceptable carrier(s). The present invention further comprises pharmaceutical compositions containing (a) canagliflozin, (b) one or more ACE inhibitors or one or more ARBs or one or more PPAR-gamma agonist and (c) one or more pharmaceutically acceptable carrier(s). Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 1.0 mg to about 500 mg of each ACE inhibitor or ARB or PPAR-gamma agonist, or any amount or range therein (when the pharmaceutical composition comprises a combination of active ingredients); and from about 25 mg to about 500 mg of canagliflozin or any amount or range therein (preferably selected from the group consisting of about 50 mg, about 75 mg, about 100 mg, about 150 mg, about 200 mg, and about 300 mg of canagliflozin). The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, transdermal, sublingual or rectal administration, or for administration by inhalation or insufflation. For preparing solid compositions such as tablets, the principal active ingredient are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. In certain embodiments, the two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from about 1.0 mg to about 500 mg of each ACE inhibitor or ARB or PPAR-gamma agonist, or any amount or range therein (when the pharmaceutical composition comprises a combination of active ingredients); and from about 25 mg to about 500 mg of canagliflozin (preferably 100 mg or 300 mg of canagliflozin) or any amount or range therein. The tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate. In certain embodiments the outer dosage component and the inner dosage component can include different active ingredients (e.g., the outer can include canagliflozin and the inner can include one or more ACE inhibitor(s) or one or more ARB(s) or one or more PPAR-gamma agonist(s); alternatively the outer can include one or more ACE inhibitor(s) or one or more ARB(s) or PPAR-gamma agonist(s) and the inner can include canagliflozin, and the like).

The liquid forms in which the compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating renal disorders, fatty liver disorders (for example NASH or NAFLD) and related disorders described in the present invention may also be carried out using a pharmaceutical composition comprising any of the compounds as defined herein and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, canagliflozin for the treatment of fatty liver disorders (for example, NASH or NAFLD) may be administered in a single daily dose, or the total daily dosage may be administered in divided doses of two, three or four times daily. Furthermore, canagliflozin for the treatment of fatty liver disorders (for example, NASH or NAFLD) may be administered in intranasal form, via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

Advantageously, compounds of the co-therapy of the present invention may be administered in a single daily dose, or the total daily dosage may be administered in divided doses of two, three or four times daily. Furthermore, compounds of the co-therapy of the present invention can be administered in intranasal form, via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component(s) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare certain pharmaceutical compositions of the present invention, canagliflozin, as the active ingredient, may be intimately admixed with a pharmaceutical carrier according to conventional pharmaceutical compounding techniques, which carrier may take a wide variety of forms depending of the form of preparation desired for administration (e.g. oral or parenteral). To prepare further pharmaceutical compositions of the present invention, canagliflozin and one or more ACE inhibitors or ARBs or PPAR-gamma agonists, as the active ingredients, may be intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by

Marcel Dekker, Inc., the disclosures of which are hereby incorporated by reference.

The following Example is set forth to aid in the understanding of the invention, and is not intended and should not be construed to limit in any way the invention set forth in the claims which follow thereafter.

### EXAMPLE 1

### Effect of Canagliflozin on Albumin/Creatinine Ratio as Measured in Subjects with Micro- or Macro-Albuminuria

The albumin/creatinine ratio was measured at baseline, over 12 weeks, over 52 weeks, and over 104 weeks, in subjects participating in the CANagliflozin cardioVascular Assessment Study (CANVAS), the DIA3004 clinical trial, and the DIA3009 clinical trial, respectively. (Complete protocol details for the CANVAS, DIA3004 and DIA3009 clinical trials are available on www.clinicaltrials.gov).

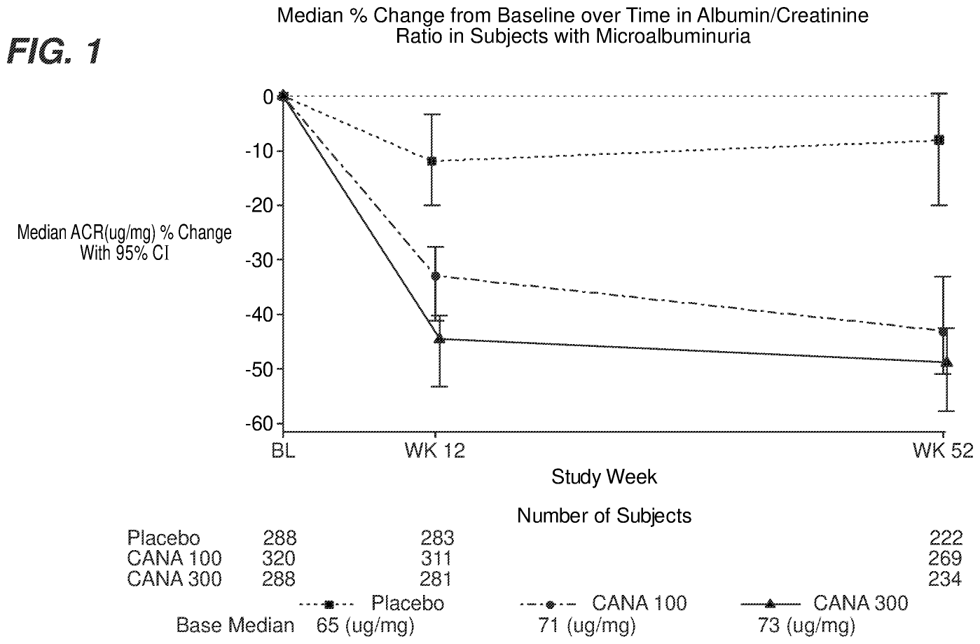

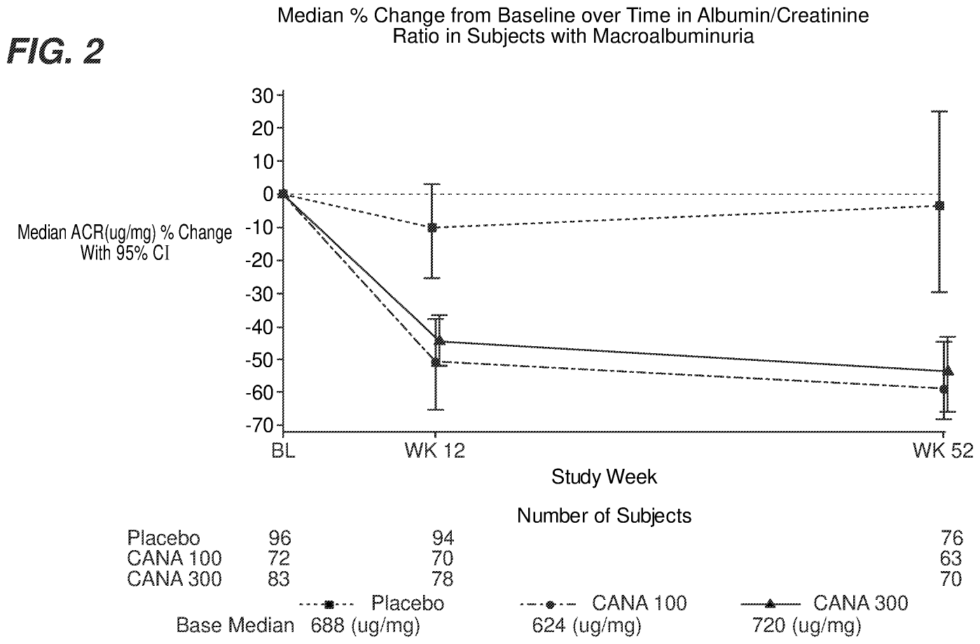

After 52 weeks of treatment in the CANVAS trial, reductions in albuminuria were seen with canagliflozin treatment in subjects with micro- and macroalbuminuria at baseline as shown in FIG. 1 and FIG. 2. In subjects with macroalbuminuria in CANVAS, the median percent change from baseline in ACR at Week 52 was -3.6% in the placebo group, −58.6% in the canagliflozin 100 mg group, and -53.3% in the canagliflozin 300 mg group. Notably this effect was seen on the background of ACEi and ARB use (82% of subjects in CANVAS were taking ACEIs or ARBs at baseline).

In a 52-week study (DIA3004) in subjects with moderate renal impairment (i.e., baseline eGFR 30 to <50 ml/min/1.73 m²), median percent reductions in albuminuria were also observed in subjects treated with canagliflozin 100 mg and 300 mg (−16.4% and −28.0%, respectively) relative to placebo (19.7%).

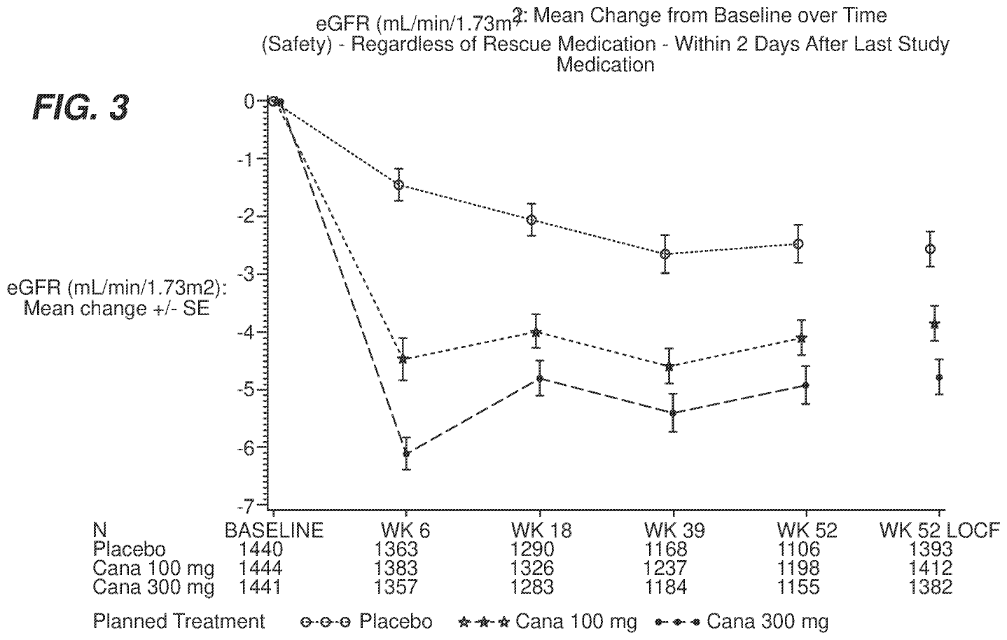

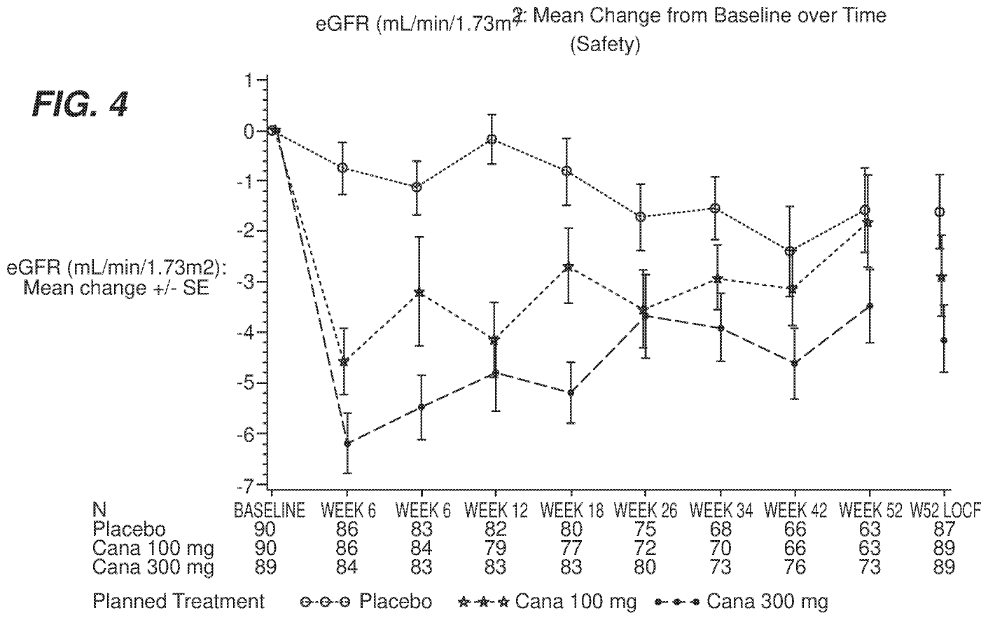

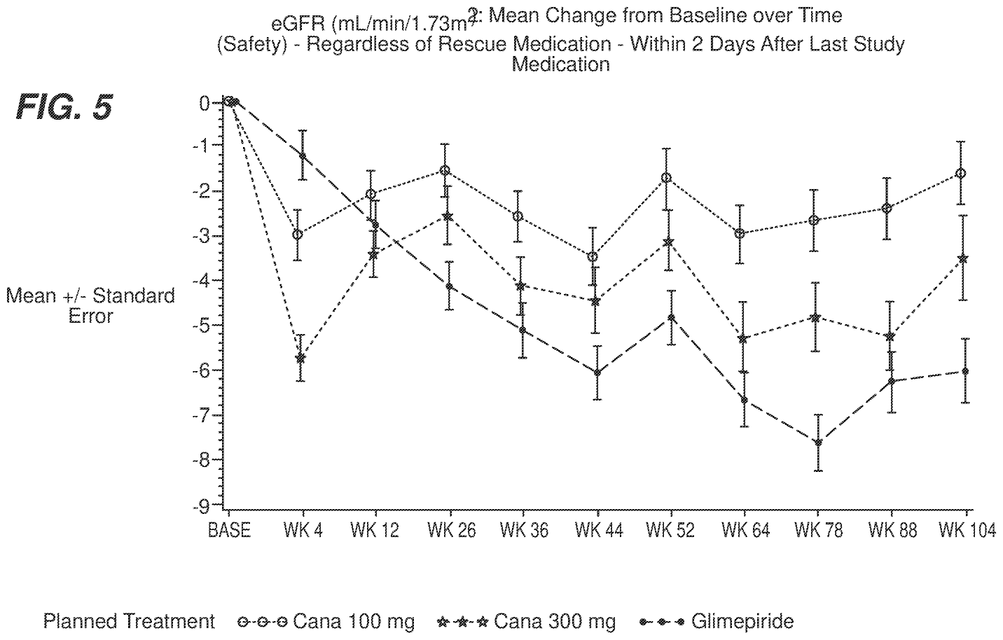

Treatment with canagliflozin was further associated with a dose-dependent, reversible reduction in eGFR that was maximal at the first post baseline visit and was either stable or attenuated with continued treatment. The time course of eGFR changes over 52 weeks in the CANVAS clinical trial is shown in FIG. 3; over a 52-week study in subjects with moderate renal impairment (in the DIA3004 clinical trial) are shown in FIG. 4; and over a 104-week period in an active comparator study (DIA3009, add-on to metformin) are shown in FIG. 5. These acute, modest declines in eGFR that do not progress and may attenuate over time are consistent with a hemodynamically mediated effect somewhat not unlike the effects seen with ACEi and ARB therapy.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method for treating, preventing, or slowing the progression of a renal disorder comprising administering to a subject in need thereof a pharmaceutical composition comprising a therapeutically effective amount of (a) canagliflozin and (b) an ACE inhibitor selected from the group consisting of benazepril, captopril, enalapril, lisinopril, imidapril and ramipril or an ARB selected from the group consisting of candesartan, irbesartan, losartan and valsartan, and (c) a pharmaceutically acceptable carrier; wherein the method decreases the urine albumin/creatinine ratio (ACR) by greater than or equal to about 30% after 52 weeks of daily administration of said composition.

2-6. (canceled)

7. The method as in claim 1 wherein the subject in need thereof has been diagnosed with or shows symptoms of one or more of the following conditions:
(a) diabetes mellitus, regardless of type;
(b) chronic kidney disease (CKD);
(c) acute renal failure (ARF);
(d) renal transplant recipients;
(e) renal transplant donors; or
(f) unilateral total or partial nephrectomized patients; or
(g) nephrotic syndrome.

8. The method as in claim 1, wherein the subject in need thereof has been diagnosed with or shows symptoms of diabetes mellitus.

9. The method as in claim 1, wherein the subject in need thereof has been diagnosed with or shows symptoms of Type 1 diabetes mellitus, Type 2 diabetes mellitus, maturity onset diabetes of the youth (MODY), latent autoimmune diabetes of adults (LADA) or pre-diabetes.

10. The method as in claim 1, wherein the subject in need thereof has been diagnosed with or shows symptoms of Type 2 diabetes mellitus.

11. The method as in claim 1, wherein the subject in need thereof has been diagnosed with or shows symptoms of Type 2 diabetes mellitus and insufficient glycemic control.

12. The method as in claim 1, wherein the subject in need thereof has been diagnosed with or shows symptoms of Type 2 diabetes mellitus and diabetic nephropathy.

13. The method as in claim 1, wherein the subject in need thereof is a patient whose measured GFR is equal to or greater than 125 mL/min/1.73 m².

14. The method as in claim 1, wherein the subject in need thereof is a patient whose measured GFR is equal to or greater than 140 mL/min/1.73 m².

15. The method as in claim 1, wherein the subject in need thereof is:
(1) an individual diagnosed of one or more of the conditions selected from the group consisting of overweight, obesity, visceral obesity and abdominal obesity; or
(2) an individual who shows one, two or more of the following signs:
(a) a fasting blood glucose or serum glucose concentration greater than about 100 mg/dL, in particular greater than about 125 mg/dL;
(b) a postprandial plasma glucose equal to or greater than about 140 mg/dL;
(c) an HbA1c value equal to or greater than about 7.0%;
(3) an individual wherein one, two, three or more of the following conditions are present:
(a) obesity, visceral obesity and/or abdominal obesity,
(b) triglyceride blood level equal to or greater than about 150 mg/dL,
(c) HDL-cholesterol blood level less than about 40 mg/dL in female patients and less than about 50 mg/dL in male patients,
(d) a systolic blood pressure equal to or greater than about 130 mm Hg and a diastolic blood pressure equal to or greater than about 85 mm Hg,
(e) a fasting blood glucose level equal to or greater than about 100 mg/dL; or
(4) an individual with obesity.

16. The method as in claim 1, wherein the canagliflozin is present as a crystalline hemihydrate.

17. The method as in claim 1, wherein the canagliflozin is administered in an amount in the range of from about 100 to about 300 mg.

18. (canceled)

19. The method as in claim 1, wherein the ACE inhibitor is selected from the group consisting of enalapril, imidapril, lisinopril and ramipril.

20. (canceled)

21. The method as in claim 1, wherein the ARB is selected from the group consisting of irbesartan and losartan.

22-69. (canceled)

70. The method as in claim 1 wherein the administration of the composition decreases the albumin/creatinine ratio (ACR) by greater than or equal to about 50%.

71. The method as in claim 1 wherein the administration of the composition decreases the albumin/creatinine ratio (ACR) in a range of from about 30% to about 90%.

72. The method as in claim 1 wherein the administration of the composition decreases the albumin/creatinine ratio (ACR) in a range of from about 30% to about 70%.

73. The method as in claim 1 wherein the administration of the composition decreases the albumin/creatinine ratio (ACR) in a range of from about 30% to about 90%.
